(19) United States
(12) Patent Application Publication
(10) Pub. No.: US 2018/0271136 A1
(43) Pub. Date: Sep. 27, 2018

(54) LOW-LECTIN DIET SYSTEM AND METHOD FOR IMPROVING HEALTH

(71) Applicant: James E. Beecham, West Palm Beach, FL (US)

(72) Inventor: James E. Beecham, West Palm Beach, FL (US)

(21) Appl. No.: 15/885,335

(22) Filed: Jan. 31, 2018

Related U.S. Application Data
(62) Division of application No. 15/619,372, filed on Jun. 9, 2017.
(60) Provisional application No. 62/601,430, filed on Mar. 21, 2017.
(60) Provisional application No. 62/601,920, filed on Apr. 5, 2017.
(60) Provisional application No. 62/602,843, filed on May 8, 2017.

(51) Int. Cl. A23L 33/00; A23L 5/10; A23L 3/10; A23L 33/16; A23L 33/15; A21D 13/40; A23L 17/00; A61K 36/185; A61K 33/18; A23L 19/00

(52) U.S. Cl. CPC A23L 33/40; A23V 2002/00; A23L 5/10; A23L 3/10; A23L 33/16; A23L 33/15; A23L 19/09; A21D 13/40; A23L 17/00; A23L 33/30; A61K 36/185; A61K 33/18

(57) ABSTRACT

A low-lectin diet system comprising a terminology-marked food package configured for enabling coordinated efficiency in healthful food selection during shopping enabling a shopper to quickly identify said package as containing low lectin-antigenicity food contents, and wherein said food package is visibly marked externally with coordinated terminology validly designating food contents as of relatively low lectin-antigenicity.

### CROSS-REFERENCE TO RELATED APPLICATIONS

The present application is a divisional application claiming priority to U.S. patent application Ser. No. 15/619,372 which claims priority to U.S. Provisional Application Ser. No. 62/604,254 filed on Mar. 16, 2017 entitled “Scratch-card and Health”, U.S. Provisional Application Ser. No. 62/601,430 filed on Mar. 21, 2017 entitled “Meal system comprising Iodine and gaming”, U.S. Provisional Application Ser. No. 62/601,644 filed on Mar. 27, 2017 entitled “Building health by reducing iodophobia”, U.S. Provisional Application Ser. No. 62/601,920 filed on Mar. 16, 2017 entitled “Network for iodine supplementation”, and U.S. Provisional Application Ser. No. 62/602,843 filed on May 8, 2017 entitled “low-inflammatory/low-hunger food combinations”, the disclosures of which are hereby incorporated in their entirety at least by reference.

### BACKGROUND OF THE INVENTION

### 1. Field of the Invention

The present invention generally relates to food science, and more particularly to a low-lectin diet system and method for improving health.

### 2. Description of Related Art

It is well known that people today face many diseases, aches and pains as well as excess hunger. One aspect of the problem has to do with fast food being readily available on-the-go, while low in nourishment and inflammatory, so it increases hunger over the day rather than satisfying hunger for hours. As a result, people overeat, get obese and develop many diseases of inflammation such as arthritis, heart disease, stroke, cancer and other diseases. Another aspect of the problem of non-nourishing food choices is that excess hunger, as with ‘empty calories’, can lead to associated nutrient deficiency and conditions such as brain fog and the like. Further, foods high in lectin can lead to detrimental effects on a person's immune system and diminished overall health. Consequently, a low-lectin diet system and method for improving health is needed allowing a more convenient means for a member of the public to identify nourishing food combinations, and to be enabled to purchase, conveniently carry, and timely consume such nourishing food combinations on a regular basis. Further, when people are healthy,they are more efficient at work, which increases productivity and brings economic prosperity.

### BRIEF SUMMARY OF THE INVENTION

In one embodiment of the present invention low-lectin diet system is provided, comprising at least one meal configured to promote health, reduce hunger, minimize pains and inflammation, such that the at least one meal comprises: (a) an ingredient mixture including an egg yolk, a fish component, and a veggie mixture; and (b) at least one low-lectin baked good; at least one snack consisting of a dark chocolate; a first at least one beneficial supplement; a second at least one beneficial supplement; and a literature component.

In one embodiment, the fish component is one sardine. In one embodiment, the veggie mixture is guacamole, wherein the ingredient mixture is mixed such that the one sardine is not recognizable. In one embodiment, the veggie mixture includes vinegar, spinach, carrot, onion, garlic, and a Himalayan salt mixture, wherein the Himalayan salt mixture includes potassium citrate. In another embodiment, the at least one low-lectin baked good is at least one sorghum cracker. In one embodiment, the at least one sorghum cracker is comprised of: (a) one part tapioca flour; (b) three part sorghum flour; (c) two parts coconut flour; (d) one part ghee; (e) one part olive oil; and (f) salt and baking power. In yet another embodiment, the first at least one beneficial supplement is an iodine supplement having a dosage amount of 12.5 mg. In one embodiment, the first at least one beneficial supplement is at least one Brazil nut. In one embodiment, the second at least one beneficial supplement is selected from the list of: vitamin D3 in coconut oil, vitamin C, vitamin K2 in medium chain triglycerides oil, zinc, magnesium, and a butyrate. In yet another embodiment, the at least one meal, the at least one low-lectin baked good, and the at least one snack combination equals at least 40% of a daily amount of calories consumed by the user. In one embodiment, the at least one meal is provided to the user raw. In one embodiment, the at least one meal is cooked and preserved in a container and co-packaged with the at least one baked good, the first at leak one supplement, and the second at least one supplement, wherein the co-packaged container is offered for sale. In another embodiment, the at least one cracker is separate from the ingredient mixture of the at least one meal, ensuring the at least one low-lectin baked good remains dry and intact. In one embodiment, the container comprises a plurality of sections, and each component of the system is separately wrapped and placed in a section of the plurality of sections, and the literature component is located on the outside of the container. In one embodiment, the literature component includes an advertisement referencing health-promoting concepts relating to the components of the system and low-lectin terminology.

In another aspect of the invention, a method for improving health, comprising steps:(a) providing a meal having fresh ingredients consisting of an egg yolk, a sardine, and a veggie mixture; (b) mixing the fresh ingredients together; (c) providing the mixed fresh ingredients to a user with at least one low-lectin baked good; (d) providing a snack consisting of a dark chocolate; (e) providing a first at least one beneficial supplement; (f) providing a second at least one beneficial supplement; and (g) providing literature referencing low-lectin terminology and health-promoting concepts relating to the steps of the method.

In one embodiment, the first at least one beneficial supplement is an iodine supplement having a dosage amount of 3 mg to 15 mg. In another embodiment, the literature includes educational facts, wherein the educational facts of the first at least one beneficial supplement include the benefits of the iodine supplement and the visual signs of iodine deficiency. In one embodiment, in step (c), the mixed fresh ingredients are raw and the at least one baked good is at least one sorghum cracker and the mixed fresh ingredients are configured to be consumed with the at least one sorghum cracker, either by a spreading or a dipping technique.

In yet another aspect of the invention, a system for improving health is provided comprising: a co-packaged diet combination including a meal, a snack, and at least one supplement; an advertisement including a plurality of health-promoting facts about the diet combination, wherein the plurality of health-promoting facts include low-lectin terminology.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a flow diagram of a low-lectin diet system and method for improving health according to one embodiment of the present invention.

FIG. 2 is a flow diagram of a low-lectin diet system and method for improving health according to one embodiment of the present invention.

FIG. 3 is an exemplary packaging structure for the diet of FIG. 2.

### DETAILED DESCRIPTION OF THE PREFERRED EMBODIMENT

The following description is provided to enable any person skilled in the art to make and use the invention and sets forth the best odes contemplated by the inventor of carrying out his invention. Various modifications, however, will remain readily apparent to those skilled in the art, since the general principles of the present invention have been defined herein to specifically provide a low-lectin diet system and method for improving health.

It is a particular advantage of the present invention to provide a diet comprising a food combination including at least one low-lectin food element. A lectin is one of a class of proteins in foods that bind specifically to certain sugars, consequently, when consumed by a person, the lectin attaches to sugar molecules within the body. This can lead to detrimental effects on the person's immune system and diminished overall health. One example of well-known lectin molecules in food, among the dozens of harmful antigenic lectins known, is wheat flour containing gluten and wheat germ agglutinin. Other high-lectin foods, specifically excluded from the diet include but are not limited to tomatoes seeds/skin, corn hull/seed coat, and lectin-rich/poorly processed beans. Each of these excluded foods include potentially disease-causing lectins. Of the grains, low-lectin or lectin-free grains are relatively rare and comprise millet and sorghum. Some lectins, having been in human diet for many centuries, are of low potential to elicit said detrimental immune response. This occurs apparently because the human immune system has been able to, over the centuries, in effect accept said lectin as non-immunogenic, not requiring an inflammatory response which might otherwise damage and cause pain and potentially excess hunger. Another particular advantage of the present invention is a method of advertising health-promoting foods included and co-packaged with the low-lectin diet, wherein the advertising helps users identify the health-promoting foods which can be consumed to improve their health. In one embodiment, the advertising includes instructions and advice regarding the low-lectin diet, including but not limited to information about lectin, foods to avoid, and recommended supplements. This will be discussed in greater detail below

Applicant's publication “Autoimmune Disease: Budget-buster or Englightened Solutions?” published Apr. 4, 2017 hereby incorporated in its entirety at least by reference, discusses the success of reducing autoimmune disease in patients by teaching them to avoid lectin-containing foods. Therefore, the advertising, instructions, and advice is a critical aspect of the present invention, and improves user health especially in situations where the user strays from the low-lectin diet provided. Consequently, a useful wording, such a “low-lectin” included in the advertising allows the user to identify health-promoting foods and/or supplements and consume the foods and/or supplements to improve their health. In one embodiment, literature is defined as any advertisement, instruction, advice, or written component related and/or co-packaged with the present invention.

In one embodiment, low-lectin/low-lectin antigenicity food elements may include but are not limited to almond flour, popped sorghum grains, millet flour, coconut flour, arrowroot flour, cassava flour, tapioca flour, avocado, cacao, coconut, ghee, goat milk, inulin, marine collagen, olive oil, salt, tempeh, sheep milk, hemp yogurt, ice cream similar to So Delicious® non-dairy no-sugar-added coconut dessert, avocado, guacamole, goat cheese, sheep cheese, casein A2 products, and coconut milk based products. In one embodiment, salt includes sea salt and. Himalayan salt, in one embodiment, the guacamole is comprised of 95% organic avocado, preferably Hass, as well as a 5% mixture of unrefined salt, onion, garlic, ascorbic acid, xanthan gum, jalapeno pepper, and citric acid.

In one embodiment, the present invention comprises a first at least one health-promoting food component consisting of a mixture of at least two of: a natural food comprised of predominately fat, a natural food comprised of predominately protein, a natural food comprised of predominately vegetable, and wherein predominately is at least 25%. In one embodiment, the present invention comprises a second at least one health-promoting component including a baked good containing a flour component, wherein the flour is a low-lectin flour or a lectin-free flour.

In one embodiment, the present invention comprises a food combination mixture of egg yolk, a fish component, and at least one vegetable component. In one embodiment, the fish component comprises sardines, preferably one sardine. In one embodiment, the food combination mixture further comprises a baked food component. In another embodiment, the food combination mixture further comprises vinegar. In yet another embodiment, the food combination further comprises at least one unrefined salt. In one embodiment, the food combination includes at least one cracker, wherein the at least one cracker comprises at least one of a low-lectin flour or a lectin-free flour, for instance sorghum flour. In another embodiment, the cracker includes an additional component consisting of one of following: an unrefined salt, olive oil, or ghee. In a preferred embodiment, the food combination is comprised in the form of a meal, as well known in the art. Further, the food combination is intended to be consumed during one day, wherein the food combination is co-packaged in a preferred embodiment. For instance, the baked food component is intended to be consumed and co-packaged, and/or co-recommended, and/or co-advertised with the mixture of egg yolk, a fish component, and at least one vegetable component. Similarly, the co-advertisement and/or co-recommendation is typically visible to shoppers at coincident retail point of sale of said multiple components, thus directing attention of health-conscious shoppers to health benefits of co-purchase and subsequent co-consumption of said multiple components. Similarly, the at least one said cracker is packaged and recommended to be consumed with the baked food component and the mixture of egg yolk, a fish component, and at least one vegetable component. That is, all the components of the food combination are either packaged together, or separately packaged and included together as they are intended to be consumed together, as within the same day. In the best mode of the present invention, the at least one cracker is used to consume a portion of the food combination, in a scooping or dipping motion as well known in the art.

In one embodiment, a supplement component is provided including but not limited to a iodine containing component, a magnesium supplement, a vitamin C supplement, a vitamin K supplement, a vitamin D supplement, a zinc supplement, and a butyric acid supplement. It is a particular advantage of the present invention to combine and co-package the supplement component: with the food combination. In one embodiment, the butyric acid supplement includes a dosage amount of 20 mg to 490 mg per day. In one embodiment, the iodine containing component includes a dosage amount between 2.2 mg to 15 mg. The dosage is the amount recommended to be consumed per day. The iodine dosage is actually 272% to 1364% over the United States government tolerable upper maximum limit amountof iodine to be consumed per day, of approximately 1.1 mg. This is a critical aspect of the present invention, as a diet consisting of thus-defined as high iodine has many scientifically proven benefits, including but not limited to reduction of risk of cancer, including breast cancer in woman, prostate cancer in men, as well as stomach cancer. Further benefits include a reduced risk of heart diseases, and beneficial for treatment for other diseases and conditions. The results are unexpected based on preconceived notions, and counter-intuitive to standard recommendations. Throughout the disclosure and particularly the claims, “iodine” may be interpreted and interchangeable with “iodine/iodide”.

It is a particular advantage of the present invention to provide a snack between meals. In one embodiment, between-meal snack is chocolate. Utilizing the meal/snack/meal/snack cycle within a day, the user has a plurality of benefits including reduced hunger throughout the day and reduced inflammation, wherein the reduced inflammation is experienced as a reduction in aches and pain. In one embodiment, the chocolate comprises dextrose as a sweetener base. This is in contrast to sucrose, which is typically found in chocolate. Sucrose includes glucose and fructose as well known in the art. Dextrose prevents the damage to the person's liver and kidney by forgoing the need to metabolize the fructose found in typically provided chocolate. In one embodiment, the between-meal snack is co-packaged with the previously mentioned supplement and food combination.

In one embodiment, the present invention has a daily amount of calories. In a preferred embodiment, the combination of the chocolate, the food component, the at least one cracker with or without the additional component added to the at least one cracker equals at least 40% of the daily amount of calories. This is a critical aspect to the diet, as it enables the user to receive a sufficient amount of low-inflammation foods providing a hunger-satisfying effect. A low-inflammation food is defined as a food or combination of foods which is low in substances known to promote inflammation in the majority of people. Thus inflammation promoting foods are limited, such as antigenic lectins.

The diet disclosed herein, is still beneficial even in situations where a user eats foods not disclosed, that is foods outside of the present invention. In this scenario the user will still derive health benefits including but not limited to low hunger, minimal aches and pains. However, the user should at least consume 30% of their total daily calories consisting of the present invention.

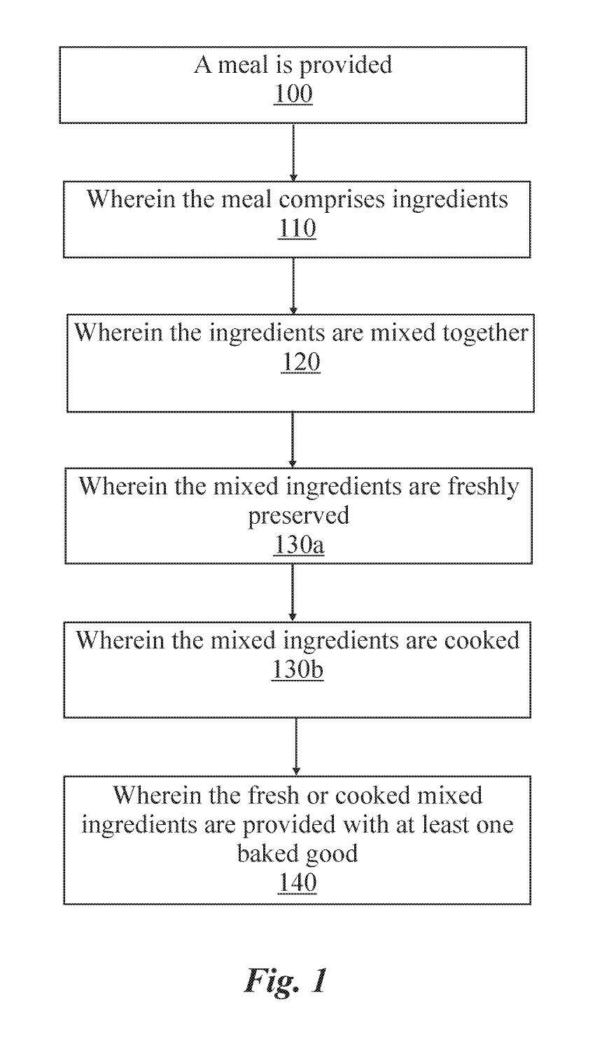

Regarding FIG. 1, one exemplary instance of a diet and method of improving health is provided. The diet and method comprises a meal 100 having ingredients 110 including an egg yolk, a sardine, and a veggie mixture. In one embodiment, the egg yolk is from 1 pastured egg. In one embodiment, the veggie mixture includes vinegar, spinach, carrot, onion, garlic, and a Himalayan salt mixture. In one embodiment, the Himalayan salt mixture includes one part in four potassium citrate. In an alternative embodiment, the veggie mixture is guacamole. In yet another embodiment, the veggie mixture is any of the vegetables or combination of vegetables as previously disclosed. In step 120, the ingredients are mixed together, such that the sardine is not recognizable. For instance, if the veggie mixture is guacamole, the meal would appear a smooth green mixture. In step 130a, the mixed ingredients are freshly preserved in a container and refrigerated, wherein the refrigerated mixed ingredients may be offered for sale in a commercial setting. Alternatively, in step 130b, the mixed ingredients are cooked and preserved in a container for storage adhering to food safety shelf life provisions well known in the art. In step 140, the fresh or cooked mixed ingredients are provided with at least one baked good. In one embodiment, the baked good is at least one cracker, wherein the mixed ingredients may be consumed with the at least one cracker, either by a spreading or dipping method well known in the art. In one embodiment, the at least one cracker is at least one sorghum cracker. In one embodiment, the meal provided may be eaten twice a day or be a supplemental or substitute to a standard meal plan. As previously disclosed it is a particular advantage of the present invention to enable a user to choose between eating the meal fresh (raw) or cooked, wherein consumed raw the user is more likely to absorb the nutrients of the meal via digestion, however raw is subject to more bacteria contamination/growth than the cooked meal as well known in the art.

In one embodiment, the ingredients and preparation of the at least one sorghum cracker is as follows: one part tapioca flour, three parts sorghum flour, two parts coconut flour, one part ghee, one part olive oil, unrefined salt to taste approximately ½ teaspoon per ½ pound of dough, and baking powder of 1 teaspoon per ¼ pound of dough. The flours are mixed thoroughly together in a first bowl, wherein the salt, and baking powder are blended in. Filtered water is added to the first bowl so that when mixed the consistency is doughy. The amount of water is dependent on the amount of dough. Next, the olive oil and ghee are placed in a second howl, wherein the second bowl is heated in a microwave for 20 seconds. Next, the heated olive oil and ghee are added to the first bowl, and blended thoroughly. Next, an oven is preheated to 225 degrees Fahrenheit. Next, a flat pan is greased with a thin layer of coconut oil, wherein the dough mixture is poured into the greased pan and then smoothed out evenly on the pan with a spatula. In one embodiment, the dough mixture should be approximately ⅛ inch thick across the pan bottom. If making bigger batches, multiple pans may be used. Finally, the dough mixture is baked at 225 degrees Fahrenheit for 30 minutes, then at 400 degrees Fahrenheit for 20 minutes, or when the mixture is brown and crisp.

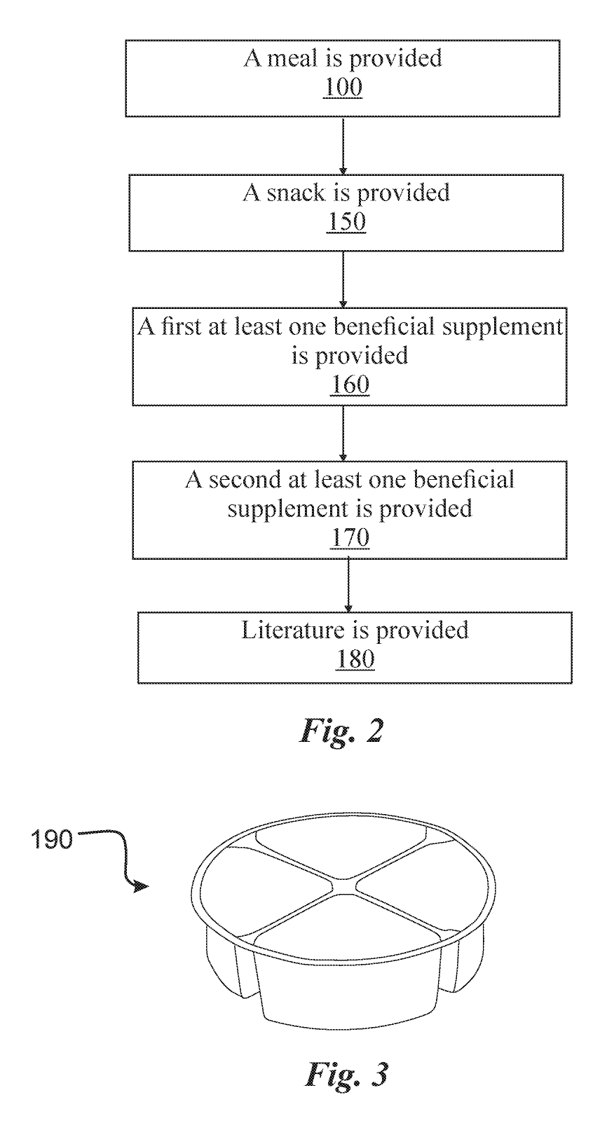

Regarding FIG. 2, the diet and method of improving health further provides a snack 150 between meals 100 and a first at least one beneficial supplement 160. In one embodiment, the snack is a dark chocolate consisting of at least 85% cocoa and a sweetener, wherein the sweetener is, optionally, a glucose sprinkle having no fructose component. In one embodiment, the sweetener is dextrose. In one embodiment, the first at least one beneficial supplement includes a 12 mg Iodoral® tablet. In one embodiment, the first at least one beneficial supplement includes at least one Brazil nut. It is a particular advantage of the present invention to include at least one Brazil nut, since Brazil nuts contain selenium, a mineral having a plurality of health benefits, including but not limited to increasing immunity, decreasing inflammation, maintaining a healthy metabolism, and regulating thyroid functions. Regarding thyroid functions, selenium is needed in the human body for activating thyroxine (T4) into triiodothyronine (T3) via activity of a de-iodinating enzyme for which selenium is a co-factor. In another embodiment, the diet and method of improving health further provides a second at least one supplement 170 selected from the list of: vitamin D3 in coconut oil, vitamin C, vitamin K2 in medium chain triglycerides oil, zinc, magnesium, and a butyrate.

In one embodiment, the diet and method of improving health provides literature 180 to the user, wherein the literature includes advice and information regarding the previously discussed foods and supplements provided in the diet system and method. In one embodiment, details of the advice include hydration advice. Specifically, the hydration advice, with approval from the user's medical doctor, the user is advised to eat a meal and/or snack and then drink a cup of water. In one embodiment, the cup of water includes salt comprised of 1.5 grams salt that is 75% unrefined salt such as, sea salt or Himalayansalt and 25% potassium citrate. Over the course of the day, the user is advised to typically consume 3 cups of such salted water. In one embodiment, the salt is co-packaged and included with the components of the system. In one embodiment, the instructions include meal preparation directions and guidelines. In yet another embodiment, the instructions include supplement facts and education related to the at least one beneficial supplement, wherein the education describes deficiencies and how the user might behave and appear under a deficiency, such as an iodine deficiency. For example, a person with an iodine deficiency displays visual signs including but not limited to thin lateral eyebrows, periorbital edema, dry coarse and sparse hair, and a puffy dull face with dry skin. Further education includes possible side effects from taking an iodine supplement such as the symptoms from flushing out bromine/bromide from the human body system which may include headaches.

Regarding FIG. 3, method of improving health further provides inventive packaging 190. In one embodiment, the at least one cracker is sufficiently separate from the meal, snack, the first at least one supplement, and the second at least one supplement. This is to ensure the at least one cracker remains dry and intact. It is a particular advantage of the present invention, that each component of the diet, in at least sonic embodiments, is wrapped separately, such as in a four part container as well known in the art.

Although the invention has been described in considerable detail in language specific to structural features and or method steps, it is to be understood that the invention defined in the appended claims is not necessarily limited to the specific features or steps described. Rather, the specific features and steps are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention. For example, the baked good of low-lectin antigenicity comprised in a embodiment of instant invention can be of a number of common configurations including but not limited to a slice of bread, such as bread slice configured as a sandwich with low-lectin-antigenicity spread at center, such as a chip, a crumble, a dumpling with an egg yolk, fish, and veggie filling or other low-lectin-antigenicity filling or the like. Furthermore, an alternative embodiment may comprise a baked good of low-lectin composition, such as a sweet cookie, and a filling or dip comprised of a low-lectin antigenicity yogurt-style composition or coconut ice cream with low-lectin-antigenicity ingredients within a low-lectin ‘ice-cream’ sandwich, or the like.

In addition, reference to “first,” “second,” “third,” and etc. members throughout the disclosure (and in particular, claims) are not used to show a serial or numerical limitation but instead are used to distinguish or identify the various members of the group.

What is claimed is:

1. A system comprising:
a terminology-marked food package configured for enabling coordinated efficiency in healthful food selection during shopping enabling a shopper to quickly identify said package as containing low lectin-antigenicity food contents, and wherein said food package is visibly marked externally with coordinated terminology validly designating food contents as of relatively low lectin-antigenicity.

2. The system of claim 1, wherein said low lectin-antigenicity food contents are minimized in content of casein A1.

3. The system of claim 1, wherein said low lectin-antigenicity food contents are minimized in content of wheat germ agglutinin.

4. The system of claim 1, wherein said low lectin-antigenicity food contents are minimized in content of tomato skin and seed.

5. The system of claim 1, wherein said low lectin-antigenicity food contents are minimized in content of corn.

6. The system of claim 1, wherein said low lectin-antigenicity food contents are minimized in content of brown rice.

7. The system of claim 1, wherein said low lectin-antigenicity food contents are minimized in content of beans and of non-sweet potato.

8. The system of claim 1, wherein said terminology validly reflects the relatively low lectin nature and relatively low lectin-antigenicity nature of said low lectin-antigenicity food contents of said terminology-marked food package having a relatively minimized content of casein A1, gluten, wheat germ agglutinin, corn, beans, and non-sweet potato.
